(19) United States
(12) Patent Application Publication
(10) Pub. No.: US 2020/0297904 A1
(43) Pub. Date: Sep. 24, 2020

(54) SUCTION CATHETER ASSEMBLIES

(71) Applicant: SMITHS MEDICAL INTERNATIONAL LIMITED, Ashford (GB)

(72) Inventor: Stephen James Field, Canterbury (GB)

(73) Assignee: SMITHS MEDICAL INTERNATIONAL LIMITED, Ashford (GB)

(21) Appl. No.: 16/651,433

(22) PCT Filed: Aug. 30, 2018
(86) PCT No.: PCT/GB2018/000116

§ 371 Date: Mar. 27, 2020

(30) Foreign Application Priority Data
Oct. 20, 2017 (GB) 1717237.0

(51) Int. Cl. A61M 1/00

(52) U.S. Cl. CPC A61M 1/0064; A61M 2210/1032; A61M 1/0066; A61M 1/0035

(57) ABSTRACT

A dual-lumen closed-system suction catheter assembly has a handpiece (2) at its machine end coupled to a suction source (27) and a source (37) of irrigating liquid. The handpiece has a pistol grip shape with a suction control (30) on its upper part (22) that can be operated by the thumb to open a valve (28) in line between the suction lumen (10) of the catheter (1) and the suction source (27). The flow of irrigating liquid to the catheter (1) is controlled by an irrigation valve (38) in the handpiece that is actuated by a trigger (50) when squeezed by the fingers of the same hand.

This invention relates to suction catheter assemblies of the kind including a suction catheter having a first lumen for suctioning and the assembly including a second lumen for supplying irrigating liquid to the patient end of the assembly.

Suction catheter assemblies may be used to remove secretions from within a tracheal tube or the respiratory passages of a patient. They are also used in other applications. Suction catheter assemblies may be of the closed-system kind in which the catheter is enclosed within a flexible envelope. Such assemblies have a manifold at the patient end with a sliding seal through which a suction catheter can be advanced and withdrawn. The flexible envelope is joined at one end to the manifold and encloses the catheter along its length. The other end of the envelope and the catheter are joined with a rear end housing including a suction control valve and a connector. The connector connects the catheter to a suction source; the valve enables the clinician to control the suction applied by the catheter. In some suction catheter assemblies the catheter has two lumens, one being used for suctioning and the other being used to supply irrigating or lavage liquid along the catheter to its patient end to aid in removing secretions from the tracheal tube.

Examples of closed-system suction catheter assemblies are described in U.S. Pat. Nos. 5,269,768, 5,300,043, 4,569,344, 4,638,539, 4,872,579, 5,167,622, 5,779,687, 5,325,850, 5,490,503, 5,419,769, 5,460,613, 5,349,950, GB2394761, GB2400160, U.S. Pat. Nos. 6,109,259, 6,227,197, EP801577B, WO96/09082, EP1239907B, EP1478424B, U.S. Pat. No. 6,588,427, EP1620148B, US2004/0221852, EP1911482A, EP1795217A, US2007/0282250, WO2007/143502, US2008/0188833, U.S. Pat. Nos. 6,227,200, 6,543,451, EP1239909B, U.S. Pat. No. 6,602,219, EP1347798, WO02/49680, U.S. Pat. No. 6,609,520, WO/055143, U.S. Pat. Nos. 6,805,125, 6,923,184, 7,021,313, 7,191,782, WO2004/101045, U.S. Pat. No. 7,263,997, WO2004/103448, WO00/15276, EP637257B, EP1113835B, EP1210957A, EP1237612B, U.S. Pat. No. 7,152,603, EP1267957B, U.S. Pat. No. 6,978,783, US2004/0007236, US2005/0211253, US2005/0211245, US2005/0235987, U.S. Pat. No. 7,059,322, WO2004/032817, US2008/0135051, U.S. Pat. Nos. 4,836,199, 4,850,350, 4,967,743, 5,025,806, 5,083,561, 5,220,916, 5,215,522, 5,255,676, 5,277,177, 5,309,902, 5,333,606, 5,343,857, 5,487,381, 5,513,628, 5,791,337, EP1343552A, WO02/49699, U.S. Pat. No. 6,612,304, EP1322371A, WO02/28463, U.S. Pat. No. 6,629,530, WO02/051485, U.S. Pat. No. 6,769,430, EP1330284, WO02/36191, U.S. Pat. No. 6,886,561, WO2004/034946, U.S. Pat. No. 7,188,623, WO2006/014431, U.S. Pat. No. 7,341,059, WO2005/094925, WO2006/103233, WO2007/030388, WO2009/003135, U.S. Pat. Nos. 4,838,255, 5,107,829, 5,133,345, 5,642,726, 6,702,789, 7,458,955, 7,273,473, 5,139,018, 4,327,723, 4,515,592, 6,099,519, EP695556B, U.S. Pat. Nos. 5,065,754, 5,730,123, 5,207,220, 5,309,903, 7,086,402, 7,597,686, 7,726,315, WO11020985 and GB2468946. Closed system suction catheters are available from various manufacturers including Smiths Medical, Kimberley Clark, Covidien and Viasys.

One problem with conventional dual-lumen catheter assemblies is that the arrangement of the suction control valve and the irrigation port to which the source of lavage fluid is connected are difficult to be used single-handed. Usually, the clinician holds the suction control valve in one hand and manipulates the suction catheter with his or her other hand while an assistant connects a lavage saline phial to the irrigation port and squeezes the phial to administer the lavage fluid. The small size of the rear end housing makes it difficult for the clinician and assistant both to grip and manipulate the housing at the same time, making the procedure awkward. It is important to control the amount of lavage liquid that is dispensed carefully because, if too much is given too quickly it might not all be removed by the suctioning and could flow down the tracheal tube into the respiratory passages. In effect, washing secretions from the tube into the lungs.

It is an object of the present invention to provide an alternative suction catheter assembly.

According to the present invention there is provided a suction catheter assembly of the above-specified kind, characterised in that the assembly includes a handpiece towards its machine end having a first port for connection with a source of suction and a second port for connection with a source of irrigating liquid, that the handpiece includes a suction control for enabling or restricting suction applied to the first lumen and that the handpiece is arranged such that the user can both actuate the suction control and control the flow of irrigating liquid to the assembly using the same hand as holds the handpiece.

Both the first and second lumens preferably extend along the suction catheter. The handpiece may include an irrigating liquid control on the hand piece that can be actuated by the same hand as holds the handpiece. The source of suction and the source of irrigating liquid may both be external of the handpiece. The handpiece preferably has a pistol grip shape with a main body portion that is gripped between the palm and fingers and an upper part on which the user can place his thumb, the suction control being actuable by the thumb on the upper part and the irrigating liquid control being actuable by the fingers of the same hand on the main body portion. The irrigating liquid control may be in the form of a pump actuator arranged to pump liquid from a liquid reservoir to the second port. Alternatively, the irrigating liquid control may be in the form of a valve arranged to control flow of irrigating liquid from a source of liquid at pressure to the second port. The source of irrigating liquid may include a compressible reservoir mounted on the handpiece such that flow of irrigating liquid to the second port can be controlled by compressing the reservoir with the same hand as holds the handpiece. The assembly may include a hinged lever extending along the compressible reservoir such that the reservoir can be compressed to dispense irrigating liquid from the reservoir by squeezing the lever. The assembly preferably includes a patient fitting including a wiper seal through which the patient end of the catheter can be extended and retracted, and that the assembly includes a flexible sleeve extending along the outside of the catheter and attached at one end with the patient end fitting and at its opposite end with the handpiece so that the catheter can be manipulated through the sleeve.

Three examples of suction catheter assemblies according to the present invention will now be described, by way of example, with reference to the accompanying drawings, in which:

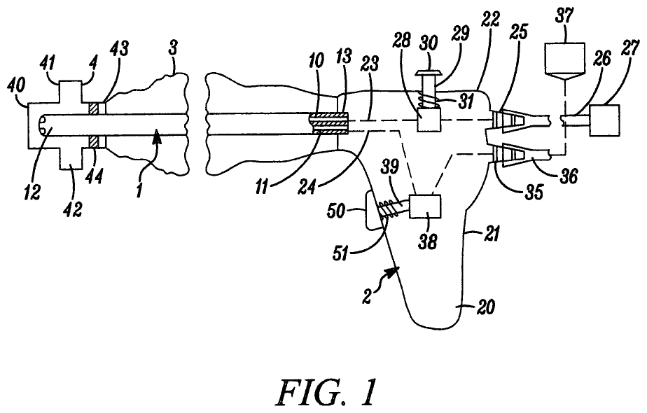

FIG. 1 is a partly sectional side elevation view of a first form of assembly;

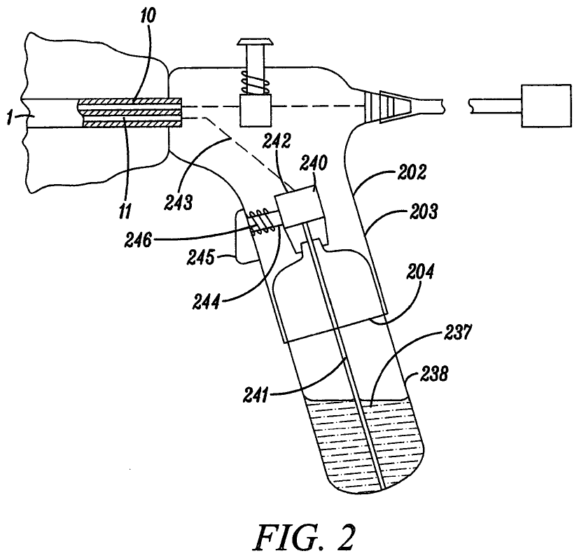

FIG. 2 is a partly sectional side elevation view of a second form of assembly;

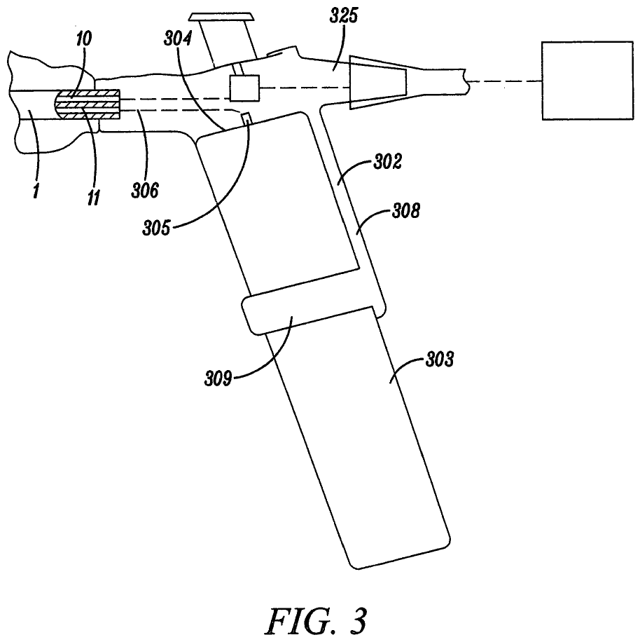

FIG. 3 is a partly sectional side elevation view of a third form of assembly; and

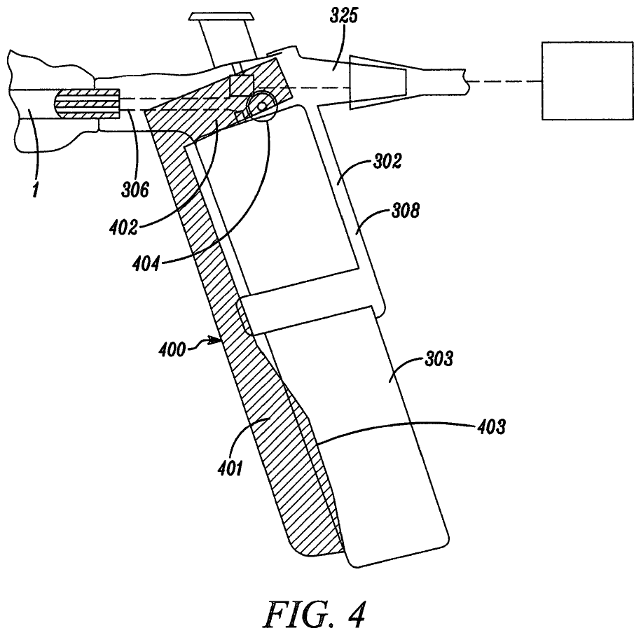

FIG. 4 shows a modification of the assembly of FIG. 3.

With reference first to FIG. 1 there is shown a first form of closed-system suction catheter assembly including a suction catheter 1 attached at its machine end with a handpiece 2. The suction catheter 1 is of the dual-lumen kind, having a suction lumen 10 and an irrigating lumen 11 extending along its length and opening at both the patient end 12 of the catheter and at its machine end 13. The catheter 1 is enclosed along its length in a flexible, transparent plastics sleeve or envelope 3 through which the catheter can be gripped and manipulated. The sleeve 3 is sealed at its machine end with the handpiece 2 and at its opposite, patient end with the rear, machine end of a patient fitting in the form of a manifold 4. The manifold 4 has a cross-shape configuration with a patient end port 40 adapted to fit onto a 15 mm coupling at the machine end of a tracheal tube (not shown), two side ports 41 and 42 adapted to be connected with a breathing system, and the machine end port 43 to which the envelope 3 is sealed. The machine end port 43 includes a wiper seal 44 through which the patient end of the catheter 1 can be advanced and retracted. As so far described the assembly is conventional.

The assembly differs from conventional assemblies in that its handpiece 2 is arranged such that the user can control both suction and the flow of irrigating liquid to the catheter using the same hand as holds the handpiece. The handpiece 2 has an outer housing 20 of a moulded plastics that is shaped into a pistol grip with a main body portion 21 that can be gripped between the palm and four fingers of the hand in a generally upright position and allowing the user's thumb to be placed on the upper part 22 of the housing. The machine end 13 of the suction catheter 1 is located inside the housing 20 of the handpiece which has a suction flow path 23 and an irrigating flow path 24, both of which can be provided by respective lengths of tubing.

The suction flow path 23 extends at one end from the suction lumen 10 at the machine end 13 of the suction catheter 1 to a suction port 25 projecting from the outside of the housing 20 on the opposite side from the suction catheter. The suction port 25 is tapered and ribbed so that one end of flexible suction tubing 26 can be pushed and retained on the port. The opposite end of the suction tubing 26 connects with a suction source, external of the handpiece 2, indicated generally by the numeral 27, which can be of any general kind, such as including a suction drainage bottle connected with a hospital suction supply and including conventional filters and overfill prevention valves or the like. The suction flow path 23 also includes a flow control valve 28 positioned along it that is normally closed, preventing suction being applied to the catheter 1, but can be manually opened to enable flow along the suction flow path. The suction control valve 28 is coupled with a manually displaceable rod 29 projecting through the upper side 22 of the housing 20 and terminated by an enlarged thumb pad 30. The rod 29 is urged upwardly and outwardly by a spring 31 in the housing 20. When the user presses down with his thumb on the pad 30 the rod 29 is pressed down and in against the action of the spring 31, thereby actuating the valve 28 to cause it to open and connect the suction source 27 with the suction catheter 1, This allows aspirated material to flow through the catheter 1, along the suction flow path 23 and valve 28 in the housing 20 and through the suction tubing 26 to the suction source 27. When the user releases his thumb the suction valve actuator rod 29 is moved up by the spring 31 to its outer, neutral or rest position.

The irrigating flow path 24 extends from the irrigating lumen 11 of the catheter 1 at one side of the housing 20 to a second, irrigating liquid port 35 projecting from the outside of the housing 20 on the opposite side from the suction catheter just below the suction port 25. The irrigating port 35 is tapered and ribbed so that one end of flexible, irrigating tubing 36 can be pushed and retained on the port. The irrigating tubing 36 extends to a source 37 of irrigating liquid, external of the handpiece 2, such as a suspended bag of saline. Alternative irrigating liquid sources are described later. The irrigating flow path 24 includes a second flow control valve 38 positioned along the length of the flow path. The irrigating flow control valve 38 is normally closed to prevent flow of irrigating liquid along the flow path 24 to the irrigating lumen 11 of the catheter 1. The irrigating flow control valve 38 is coupled with a manually-displaceable rod 39 projecting through the left-hand, forward side of the main body portion 21 of the housing 20 and is terminated by an enlarged trigger pad 50. The rod 39 is urged outwardly by a spring 51 in the housing 20. When the user places his index or middle finger on the trigger pad 50 and presses this inwardly against the action of the spring 51 it moves the rod 39 inwardly and actuates the irrigating valve 38. This causes the irrigating valve 38 to open and allow irrigating liquid to flow from the irrigating source 37 to the irrigating lumen 11 of the catheter 1.

The arrangement of this assembly enables the handpiece 2 to be gripped by the user in one hand. Suction can be controlled easily simply by pressing the suction pad 30 down with the thumb. Irrigation can also be controlled easily simply by squeezing the irrigating trigger 50 with the fingers of the same hand. In this way suction and irrigation can be controlled at the same time enabling just suction, or just irrigation, or both suction and irrigation to be applied by the catheter 1. As only one hand is needed to control suction and irrigation the user can use his other hand to manipulate the suction catheter 1 into the tracheal tube, advancing it or retracting it to ensure effective removal of any secretions.

Instead of connecting the handpiece of the assembly to a remote source of irrigating liquid via tubing, as described with reference to FIG. 1, it would be possible to mount the source of irrigating liquid directly on the assembly and supported by the handpiece itself. Arrangements for doing this are shown in FIGS. 2, 3 and 4.

In the arrangement shown in FIG. 2 the source of irrigating liquid 237 is provided by a bottle 238 of irrigating liquid and the handpiece 202 has a housing 203 that is open at its lower end 204 so that the open upper end of the bottle 238 can be fitted into the handpiece. In place of the valve described in relation to the embodiment of FIG. 1, this arrangement includes a mechanical pump 240 having an inlet wand 241 depending from the pump down into the bottle 238 close to its lower, closed end. The wand 241 provides the second, irrigating port of the handpiece 202. The pump 240 has an outlet 242 at its upper end connected by a tube 243 to the machine end of the irrigation lumen in the suction catheter. The pump 240 is driven by an actuating rod 244 extending from the pump through the front wall of the hand piece 202 and terminated in a trigger pad 245. The rod 244 is urged outwardly by a spring 246 in the housing 203. To operate the pump, and hence cause irrigating liquid to flow from the bottle 238 to the irrigating lumen 11 in the catheter 1, the trigger pad 245 is squeezed inwardly by the user's forefinger against the action of the spring 246 and is then released. This causes a measured bolus of liquid to flow along the irrigation lumen 11 of the catheter 1. The user can squeeze and release the trigger 245 repeatedly to dispense as much irrigation fluid as is needed.

The arrangement shown in FIG. 3 is similar to that shown in FIG. 2 in that the source of irrigating liquid is supported by the handpiece 302 but differs in that it lacks any pumping mechanism. Instead, the irrigating liquid is contained within a cylindrical phial 303 of a resilient material. The phial 303 is open at its upper end 304 and is connected to a port 305 within the handpiece 302. A tube 306 extends from the irrigating fluid port 305 to the irrigating lumen 11 of the suction catheter 1. The body of the handpiece 302 is modified to have an open, skeletal form where the phial 303 is supported by a narrow vertical rib 308 extending down half the length of the phial on the side remote from the suction catheter. A narrow ring formation 309 at the lower end of the rib 308 embraces the phial 303 midway along its length. The remaining surface of the phial 303 is exposed either side of the ring 309 so that it can be squeezed by gripping with the fingers so as to compress the phial and squeeze out some of its contents into the irrigating lumen 11 of the suction catheter 1.

Instead of the user directly contacting and squeezing the phial 303 with his fingers it would be possible to use a modified assembly of the kind shown in FIG. 4, which includes a lever 400 by which the phial can be squeezed indirectly. The lever 400 has an L-shape with a long arm 401 and a shorter arm 402 extending at right angles to the long arm at its upper end. The long arm 401 extends down along the front side of the phial 303 and has a protrusion 403 towards its lower end projecting towards the front side of the phial 303 where this extends below the ring 309. The short arm 402 extends rearwardly a short distance at the upper end of the phial 303 towards the suction inlet 325. The lever 400 is pivotally mounted on the handpiece 302 at the upper end of the phial 303 at a location along the short arm 402. A coil spring 404 or other resilient member is mounted between the lever 400 and the handpiece 302 to urge the lever in a clockwise sense so that the long arm 401 is urged away from the phial 303 in its natural state. When irrigation is needed the user simply squeezes the long arm 401 of the lever 400 against the action of the spring 404 to rotate the lever through a small angle anticlockwise and force the protrusion 403 against the side of the phial 303. This squeezes the phial 303 and dispenses the irrigation liquid. The lever 400 ensures that the pressure is always applied to the phial 303 at the same location regardless of where the user squeezes the lever. This may make it easier for someone with small hands to operate the assembly.

The arrangements of FIGS. 2, 3 and 4 have the advantage of avoiding the need for suction tubing to be attached to the handpiece, making the assemblies easier to manipulate without tangling. The disadvantage of these arrangements, however, is that the weight of the irrigating liquid adds to the total weight of the handpiece and the quantity of irrigating fluid is more limited, which may require replacement of the bottle or phial.

The invention is not confined to closed-system suction catheters with an outer protective envelope but could be used with exposed suction catheters. The suction catheter assemblies are not confined to those for use in tracheal tubes but could be used in other applications where both suction and irrigation is needed. The source of irrigating liquid could be provided by any conventional means, such as, for example, an elastomeric pump or a liquid container in which the liquid is forced out by gas pressure within the container. The suction catheter need not have two lumens since it could just have a suction lumen and the irrigating lumen be provided in a separate tube opening in the machine end fitting. This still enables the catheter to be irrigated when it is withdrawn but allows the outer diameter of the suction catheter to be minimised, which is especially important in paediatric applications.

1-10. (canceled)

11. A suction catheter assembly including a suction catheter having a first lumen for suctioning and the assembly including a second lumen for supplying irrigating liquid to the patient end of the assembly, characterised in that the assembly includes a handpiece towards its machine end having a first port for connection with a source of suction and a second port for connection with a source of irrigating liquid, that the handpiece includes a suction control for enabling or restricting suction applied to the first lumen and that the handpiece is arranged such that a user can both actuate the suction control and control the flow of irrigating liquid to the assembly using the same hand as the user holds the handpiece.

12. The assembly according to claim 11, characterised in that both the first and second lumens extend along the suction catheter.

13. The assembly according to claim 11, characterised in that the handpiece includes an irrigating liquid control on the handpiece that can be actuated by the same hand as the user holds the handpiece.

14. The assembly according to claim 11, characterised in that the source of suction and the source of irrigating liquid are both external of the handpiece.

15. The assembly according to claim 11, characterised in that the handpiece has a pistol grip shape with a main body portion that is gripped between the palm and fingers and an upper part on which the user can place his thumb, and that the suction control is actuable by the thumb on the upper part and the irrigating liquid control is actuable by the fingers of the same hand on the main body portion.

16. The assembly according to claim 11, characterised in that the irrigating liquid control is in the form of a pump actuator arranged to pump liquid from a liquid reservoir to the second port.

17. The assembly according to claim 11, characterised in that the irrigating liquid control is in the form of a valve arranged to control flow of irrigating liquid from a source of liquid at pressure to the second port.

18. The assembly according to claim 11, characterised in that the source of irrigating liquid includes a compressible reservoir mounted on the handpiece such that flow of irrigating liquid to the second port can be controlled by compressing the reservoir with the same hand as holds the handpiece.

19. The assembly according to claim 18, characterised in that the assembly includes a hinged lever extending along the compressible reservoir such that the reservoir can be compressed to dispense irrigating liquid from the reservoir by squeezing the lever.

20. The assembly according to claim 11, characterised in that the assembly includes a patient fitting including a wiper seal through which the patient end of the catheter can be extended and retracted, and that the assembly includes a flexible sleeve extending along the outside of the catheter and attached at one end with the patient end fitting and at its opposite end with the handpiece so that the catheter can be manipulated through the sleeve.
